(19) United States
(12) Patent Application Publication
(10) Pub. No.: US 2013/0023681 A1
(43) Pub. Date: Jan. 24, 2013

(54) STABILIZED DOXERCALCIFEROL AND PROCESS FOR MANUFACTURING THE SAME

(71) Applicants: Graham McGowan, Toronto (CA); Boris Ivanovich Gorin, Oakville (CA)

(72) Inventors: Graham McGowan, Toronto (CA); Boris Ivanovich Gorin, Oakville (CA)

(73) Assignee: ALPHORA RESEARCH INC., Mississauga (CA)

(21) Appl. No.: 13/638,673

(22) PCT Filed: Mar. 29, 2011
(86) PCT No.: PCT/CA11/50165

§ 371 Date: Oct. 1, 2012

(30) Foreign Application Priority Data
Mar. 30, 2010 (CA) 2,698,160

(51) Int. Cl. C07C 401/00

(52) U.S. Cl. 552/653

(57) ABSTRACT

1 a-hydroxy vitamin D₂ (doxercalciferol) of exceptionally high purity and stability is prepared by a process involving chromatographically purifying 1 α-hydroxyvitamin D₂ monoacetate, chemically removing the acetate protectant group from the purified product to form 1 α-hydroxyvitamin D₂, and precipitating the 1 a-hydroxyvitamin D₂ so formed from a mixed organic solvent consisting essentially of at least one C1-C6 dialkyl ether or C1-C6 alkyl ester, and at least one C5-C12 hydrocarbon.

### FIELD OF THE INVENTION

This invention relates to 1α-hydroxyvitamin D₂, also known as doxercalciferol. More particularly, it relates to processes for preparing 1α-hydroxyvitamin D₂ in especially pure form, and to the form of 1α-hydroxyvitamin D₂ which can be produced by the novel process.

1. Background of the Invention

1α-hydroxyvitamin D₂ is a known pharmaceutically active compound, useful as a vitamin supplement in human therapy. It is, however, subject to oxidative degradation, rendering it chemically unstable in the presence of oxygen and light, and at elevated temperatures commonly experienced in pharmaceutical formulation preparation.

Known methods of preparation of vitamin D derivatives such as 1α-hydroxyvitamin D₂ of high purity involve procedures involving several chromatographic purifications of intermediate compounds, and a step of irradiation with ultraviolet light at the final processing step. Such irradiation steps tend to lack specificity, so that they need to be followed by further chromatographic purification and re-crystallization of the crude material to attain purity as high as 98%.

2. Brief Reference to the Prior Art

U.S. Pat. No. 6,903,083 Knutson et al. describes a process for the synthesis of 1α-hydroxyvitamin D₂ which is reported to yield a product of at least 98% purity, and which has residual solvents of 0.5% or less, total impurities of 1.5% or less, and has no single impurity greater than 0.5% by HPLC. The product so formed is reported to have improved stability, attributed to its low impurity levels.

The patent reports that the 1α-hydroxyvitamin D₂ of this purity can be prepared by any of the known methods of synthesis. The exemplified process described therein starts from vitamin D and converts it to the cyclovitamin form, hydroxylates it at the 1α-position, re-converts the hydroxylated cyclovitamin to the cis and trans forms of the vitamin, and converts the trans form to the cis form by irradiation with ultraviolet light.

It is an object of the present invention, from one aspect, to provide a 1α-hydroxyvitamin D₂ composition of higher purity and improved stability.

It is a further object of the present invention, from another aspect, to provide a novel process for preparing 1α-hydroxyvitamin D₂ which is capable of producing the product at a purity of 99% or higher, and which does not involve UV irradiation steps.

### SUMMARY OF THE INVENTION

According to a first aspect of the present invention, there is provided stabilized 1α-hydroxyvitamin D₂ which is characterized by a purity of at least 99%, and by a degree of stability such that it exhibits no reduction in purity after storage for one month, three months and six months at 25±2° C. and 60±2% relative humidity under argon head space.

According to another aspect, the invention provides a process of preparing stabilized 1α-hydroxyvitamin D₂ of at least 99% purity, which comprises:

chromatographically purifying 1α-hydroxyvitamin D₂ monoacetate,

chemically removing the acetate protectant group from the purified product to form 1α-hydroxyvitamin D₂,

and precipitating the 1α-hydroxyvitamin D₂ so formed from a mixed organic solvent consisting essentially of at least one C1-C6 dialkyl ether or C1-C6 alkyl ester, and at least one C5-C12 aliphatic hydrocarbon.

It has been discovered that samples of 1α-hydroxyvitamin D₂ of purity 99.0% and higher exhibit unexpectedly high stability at −20° C. and even at 5° C. over extended periods of time (e.g. six months). The process of the present invention produces such highly pure, stable 1α-hydroxyvitamin D₂ directly. The penultimate intermediate in the process, 1α-hydroxyvitamin D₂ monoacetate, possesses a particular set of physico-chemical properties, notably its lipophilic nature, rendering it purifiable to a high degree using, for example, silica gel chromatography.

Following deprotection to remove the acetate protectant group, final purification of the product 1α-hydroxyvitamin D₂ takes place according to the invention by precipitation from the aforesaid mixed organic solvent system. Preferred constituents of the solvent system are tert.butyl methyl ether and heptane. Tert.butyl methyl ether (MTBE) is a solvent for the product, whereas the non-polar heptane is an antisolvent. Balancing these in the appropriate ratio (about 3:1 v/v, heptane in excess) for precipitation of 1α-hydroxyvitamin D₂ yields the highly pure, stabilized product of the invention. Thus the process of the invention may be said to be characterized by the combination of (i) purification of the penultimate intermediate, and (ii) adoption of a special mixed organic solvent system for precipitation of the final product.

### BRIEF REFERENCE TO THE DRAWING

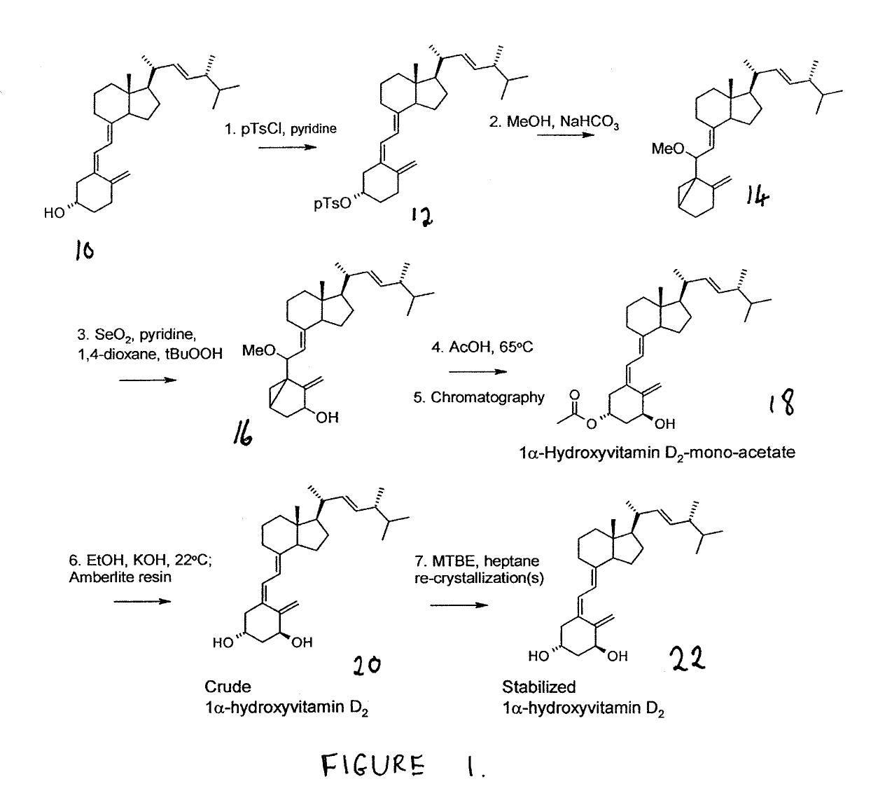

The accompanying single FIG. 1 of drawings is a diagrammatic illustration of an embodiment of the process of synthesizing 1α-hydroxyvitamin D₂ according to the invention, starting from vitamin D₂.

### THE PREFERRED EMBODIMENTS

The process as illustrated on the accompanying Figure uses vitamin D2, compound 10, as its starting material. In a first reaction step, the 3-hydroxyl group of compound 10 is activated, in this example by reaction with p.toluylsulphonyl chloride, to insert a p.tosyl leaving group, compound 12. In a second step, cycloisomerization is effected, by reaction with sodium bicarbonate in methanol, to produce cyclovitamin D2, compound 14. This is a known chemical method of effecting protection of a triene system.

Next, cyclovitamin D2 is oxidized at the allylic position by reaction with selenium dioxide, 1,4-dioxane and tert.butyl hydroperoxide acid, in pyridine.

Compound 16, 1-OH-cyclovitamin D2, is formed, which has the required 3-hydroxy group of the target compound, but is formed as a mixture of α and β epimers at the C1 position. The desired isomer for the final compound is the a epimer. It is noteworthy that no step of purification is necessary at this stage, following the selenium dioxide oxidation.

Accordingly, the next step in the process effects a cyclo-reversion and restores the triene system, by reaction with acetic acid at an elevated temperature of about 65° C. This results in the formation of 1α-OH and 1β-OH cis- and trans-vitamin D2-mono-acetates, compound 18. Chromatographic purification of this mixture through silica gel provides a similar mixture of compounds, but with a much enhanced proportion of cis-1α-OH-vitamin-D2. This reduces the amount of other isomers in the product to a level where they can subsequently be removed, substantially entirely, by recrystallization.

In the next step of the process, the mono-acetate group is removed, and the reaction mixture neutralized to remove acid species. This can be accomplished at room temperature, by base-catalyzed de-acetylation with potassium hydroxide in ethanol, followed by neutralization with Amberlite acidic resin to absorb the basic reaction products. The resulting product, compound 20, is “crude” 1α-hydroxyvitamin D2. This is purified, in a final step, by re-crystallization, one or more times, from a mixture of MBTE (solvent) and heptane (anti-solvent), at an approximate ratio of 3:1 v/v, heptane in excess. This produces the stable, highly pure (99%)1α-hydroxyvitamin D2, compound 22, of the invention.

The product of the present invention shows exceptionally good stability. In accelerated stability studies, samples of the product of purity 99% and above have exhibited no reduction in purity after 1, 3 and 6 month's storage at 25±2° C. and relative humidity 60±2% under an argon headspace. In ICH (the internationally accepted industry standard) stability studies, they show no reduction in purity after six months storage under an argon headspace at either −20±5° C. or at 5±3° C.

The invention is further described, for illustrative purposes, in the following specific experimental examples.

### EXAMPLE 1

### Activation of Vitamin D₂ as its Tosylate

### FIG. 1, Conversion of Compound 10 to Compound 12

A 3-neck RB flask fitted with mechanical stirrer, thermometer and nitrogen inlet was charged Vitamin D₂ (125 g, 0.315 mol), Compound 10, in 200 mL pyridine at room temperature; the resultant yellow solution was cooled to 0° C. and then to it was charged a solution of para-toluenesulfonyl chloride (155 g, 0.813 mol) in pyridine (425 mL) over 39 minutes. Once the addition was complete, the cooling bath was removed and the reaction allowed to warm to room temperature and agitated overnight. After this period of time, tic analysis indicated that the reaction was complete; the dark brown suspension was cooled to 0° C. over 15 minutes, and to it was charged a total of 940 mL H₂O in portions of 470 mL over 3 hours 10 minutes, and 470 mL over 26 minutes, respectively, resulting in a thick brown suspension. The mixture was agitated at 14-15° C. for 1 hour prior to being filtered; 312 mL of H₂O was used to rinse forward any residual solids and to wash the filter cake; the cake was then washed with 2 fresh portions of H₂O (312 mL each). The pale brown solids were transferred from the funnel to an evaporating dish and dried in a vacuum oven at 37° C. for 45.5 hours. 297.3 g of tan solids were obtained from this procedure. ¹ H NMR was consistent with that of tosylated vitamin D₂.

### EXAMPLE 2

### Formation of Cyclo-Vitamin D₂

### FIG. 1, Conversion of Compound 12 to Compound 14

A 3-neck 5L RB flask fitted with mechanical stirrer, reflux condenser and nitrogen inlet was charged para-toluenesulfonyl-vitamin D₂ (compound 12, 169.8 g, 0.308 mol), NaHCO₃ (196.8 g, 2.343 mol), methanol (1290 mL) and iso-propyl acetate (712 mL); the resultant tan solution was then heated to reflux. After overnight stirring at reflux, tic indicated complete consumption of starting material. The flask was fitted with a distillation head and diaphragm pump. At an internal temperature of 35° C. the solution was distilled to approximately ½ of its original volume; to the mixture was added 1,4-dioxane (1100 mL). The mixture was once again distilled to approximately ½ of its original volume before more 1,4-dioxane (1100 mL) was added followed by a final distillation to ⅓ of the original volume to afford a thick amber slurry. The slurry was agitated at room temperature with Hyflo Supercel celite (50.9 g) for 25 minutes; after this period the slurry was filtered under suction; the filter cake was washed with 2 portions of 1,4-dioxane (2×590 mL). The filtrate and washes were combined to afford a pale orange solution (943.2 g); concentration of a portion of the solution under reduced pressure to constant weight indicated a total dissolved solids of 122.1 g; ¹ H NMR was consistent with methoxy-cyclo-vitamin D₂, compound 14

### EXAMPLE 3

### Allylic Oxidation to α-Hydroxy-Methoxy-Cyclo-Vitamin D₂

### FIG. 1, Conversion of Compound 14 to Compound 16

A 3-neck 5L RB flask fitted with mechanical stirrer, thermometer and nitrogen inlet was charged with selenium dioxide (39.6 g, 0.357 mol) and 1,4-dioxane (604 mL). At room temperature, the flask was charged dropwise with tert-butyl hydroperoxide (5.0-6.0M solution in decane, 95 mL, 0.476 mol), affording a white suspension which was then agitated at this temperature for 1.5 hours. The mixture was cooled to 15° C., and to it was charged pyridine (24 mL, 0.297 mol) dropwise. After agitation at this temperature for 10 minutes, a solution of methoxy-cyclovitamin D₂ (compound 14, 122.1 g, 0.297 mo1) in 1,4-dioxane (from the solution obtained in the previous step) was added over a period of 2 hours, maintaining a temperature of 12-15° C. during the addition. The reaction was stirred at a temperature of 13-16° C. for approximately 2 hours; successive tic analyses during this period indicated that starting material had been consumed and that no further reaction had been observed. The reaction was quenched by the drop-wise addition of H2O (511 mL) and 50% w/w aqueous NaOH solution (84.3mL) over 30 minutes; the mixture was than agitated for an additional 35 minutes. the mixture was charged with iso-propyl acetate (760 mL) at room temperature and the biphasic mixture stirred for 20 minutes. After this time, the phases were separated and the lower aqueous phase extracted for 20 minutes with another portion of iso-propyl acetate (760 mL); the phases were separated, and the combined organics concentrated in vacuo to a volume of approximately 360 mL. The solution was co-evaporated with heptane (3 portions of 700 mL) to a final volume of 230 mL. The dark orange solution was agitated with a slurry of Hyflo Supercel celite (24.5 g) in heptane (179 mL) for 15 minutes at room temperature. The slurry was filtered under reduced pressure, and the cake washed with heptane (2 portions of 45 mL each). The resulting solution was concentrated under vacuum to a constant weight of 145.2 g; ¹H NMR was consistent with that of compound 16, hydroxylated cyclo-vitamin D₂ ; the product was used in the next step without further purification.

### EXAMPLE 4

### Acetolysis to Mono-Acetate-1α-Hydroxyvitamin D2

### FIG. 1, Conversion of Compound 16 to Compound 18

A 3-neck 3L RB flask fitted with mechanical stirrer, thermometer and nitrogen inlet was charged with hydroxylated cyclo-vitamin D₂ (145.2 g, 0.296 mol, compound 16) and glacial acetic acid (884 mL). The dark orange solution was stirred at an internal temperature of 65° C. for 1 hour, 20 minutes, after which time tic indicated complete consumption of starting material. The reaction mixture was transferred to a 1-neck RB flask and concentrated under vacuum until no more distillate was observed; the crude was then co-evaporated with heptane (3 portions of 720 mL) to a final volume of 460 mL. The dark orange solution was transferred back into a 3-neck RB flask, and then charged with tert-butyl methyl ether (445 mL). To the agitated solution at room temperature was charged a solution of NaHCO₃ (41 g) in H₂O (390 mL) over a period of 7 minutes; the biphasic mixture was stirred at this temperature for 20 minutes prior to being transferred to a separatory funnel. The phases were separated and the organic was agitated for 30 minutes with saturated brine solution (405 mL); the phases were separated with the aid of additional tert-butyl methyl ether (50 mL+50 mL) and saturated brine solution (40 mL) to break an emulsion. The aqueous phase and interface was extracted into tert-butyl methyl ether (200 mL); the phases were separated, and the organic phases combined and concentrated under vacuum to a volume of 420 mL. The solution was then co-evaporated with heptane (2 portions of 200 mL); additional heptane (185 mL) was then charged to give a dark orange solution (304.3 g) which was further demonstrated to have total dissolved solids content of 138.2 g.

### EXAMPLE 5

### Column Chromatography Purification of Compound 18, Mono-Acetate-1α-Hydroxyvitamin D₂

77 g (35 g by TDS) of the above crude solution of mono-acetate-1α-hydroxyvitamin D₂ was loaded onto a column of silca gel (525 g that had been previously dry-packed and conditioned with a pre-mixed solution of heptane: tert-butyl methyl ether: triethylamine, 94:4:2 v/v, 12L in total). Once loaded onto the silica bed, the column was eluted with a pre-mixed solution of heptane: tert-butyl methyl ether: triethylamine (94:4:2 v/v, 25.5L in total); after 6000 mL of fore-run was collected, 145 fractions of 135-150 mL each were collected. Fractions 51-143 were combined and concentrated under vacuum to yield 6.51 g of an orange oil.

### EXAMPLE 6

### De-Acetylation to Crude 1α-Hydroxyvitamin D₂

### FIG. 1, Conversion of Compound 18 to Compound 20

6.4 g of compound 18, mono-acetate-1α-hydroxyvitamin D₂ was suspended in degassed ethanol (58 mL) to afford a turbid orange suspension; the mixture was concentrated under vacuum to yield a pale orange oil; the resultant orange oil was re-suspended in ethanol (58 mL) and concentrated in-vacuo to a constant weight of an orange hard, sticky foam (6.0 g). This foam was re-suspended in ethanol (17.5 mL) and transferred to a 3-neck 250 mL flask fitted with stir-bar, addition funnel, thermometer and nitrogen inlet. Some solids remained undissolved—a total of 36 mL additional ethanol was added. To the orange suspension in the flask was added a solution of KOH (flakes, 0.0823 g) in ethanol (58 mL), at room temperature over 10 minutes. The orange suspension was allowed to agitate for approximately 43 hours, and was periodically checked by TLC. To the mixture was added Amberlite 1R120 Hydrogen Form resin (1.28 g, freshly washed with 2.5 mL WFI water, followed by 3 rinses of 2.5 mL ethanol and dried under vacuum to afford 0.97 g dry resin); the pH of the mixture was checked with wetted pH paper; when pH of 5 was achieved (ca. 30 minutes) the suspension was filtered, and the resin cake washed forward with degassed ethanol (2×29 mL portions), to afford a clear, dark amber solution. This solution was concentrated in-vacuo until no more condensate was observed. To the resultant amber oil was added degassed MTBE (115 mL); the solution was then concentrated in-vacuo until no more distillate was observed; this procedure was repeated twice to afford an amber/brown oily foam (5.5 g).

The brown foam obtained above was dissolved in degassed MTBE (29.2 mL) and transferred to 100 mL 3-neck RB flask fitted with stir-bar, thermometer and nitrogen inlet. With stirring, to the flask was charged degassed heptane (86 mL) dropwise over 21 minutes at room temperature; after the addition of heptane was complete, a thick, pale yellow slurry was evident in the flask. The slurry was agitated overnight at room temperature. After this time, the suspension was filtered under a blanket of nitrogen; the filtrate was used to rinse the residual solids forward. The solids were dried to constant weight in a dessicator, yielding 3.14 g of off-white solid.

### EXAMPLE 7

### Re-Crystallization of 1α-Hydroxyvitamin D₂

### FIG. 1, Formation of Compound 22

3.10 g of 1α-hydroxyvitamin D₂ from Example 6 was suspended in degassed MTBE (68.2 mL) and stirred for 30 minutes at room temperature; the mixture remained as a suspension; a total of 18 mL additional MTBE was added to achieve dissolution; at this point the mixture was filtered to remove particulate matter, and then concentrated in-vacuo to a weight of 43.87 g. The solution was transferred in degassed MTBE (13.1 mL) to 500 mL 3-neck RB flask fitted with stir-bar, thermometer, addition funnel and nitrogen inlet. With agitation at room temperature, the flask was charged with degassed heptane (186 mL) dropwise over 23 minutes; white solids were observed to precipitate from solution after approximately ⅔ of the heptane addition. The thick beige suspension was agitated at room temperature overnight. After this period, the suspension was filtered under a blanket of nitrogen; the solids were rinsed forward with ca. 5 mL of the filtrate. The resultant white solids were dried in a dessicator, affording 2.06 g of product, 1α-hydroxyvitamin D₂.

### EXAMPLE 8

### HPLC Analysis of Samples of 1α-Hydroxyvitamin D₂

Samples of material generated by the aforementioned procedure were quarantined, stored and subjected to 2 stability studies:

A. Accelerated storage study

B. Long term stability

Both studies employed ICH-compliant stability chambers for controlled storage, and the following HPLC method for analysis of samples:

HPLC Detector/wavelength: Photo Diode Array Detector/190-400 nm

Column: Waters SUNFIRE C18, 4.6 by 150 mm, 3.5 μm

Column/sample Temperature: 25° C./5° C.

Flow Rate/injection volume: 1.2 mL per min/10.00 μL

Run Time: 55 min

Sample concentration: 1 mg/mL

Diluent: Water: DCM: MeOH: ACN (10: 5: 10: 75)

Eluent: A (H2O); B (ACN); C (MeOH)

Gradient: time (% A: % B: % C)
t=0 (30: 60: 10)
t=45 (0: 90: 10)
t=46 (30: 60: 10)
t=55 (30: 60: 10)

A summary of the storage protocols and results is presented below:

A. Accelerated Storage Study

Samples were subjected to the following conditions: 25±2° C./60±2% R.H., Argon headspace. Samples were stored in ICH-compliant stability chambers, sampled at 1, 3 and 6 months, and analyzed using the described HPLC method.

a. Results—HPLC Purity

Samples of 1α-hydroxyvitamin D₂ analyzed to have initial (t=0) HPLC a/a purity of >99.0% a/a were shown to have no reduction in purity below 99.0% under the conditions of accelerated storage, at any of the time-points of 1, 3 and 6 months.

b. Results—HPLC Assay

Samples of 1α-hydroxyvitamin D₂ analyzed to have initial (t=0) HPLC w/w assay of >99.0% w/w were shown to have no reduction in HPLC assay below 99.0% w/w, under the conditions of accelerated storage, at any of the time-points of 1, 3 and 6 months.

B. Long Term Stability Study

a. Protocol

Samples were subjected to the following conditions: 5±3° C. Argon headspace, −20±5° C., Argon headspace. Samples were stored in ICH-compliant stability chambers, sampled at 1, 3, 6 and 9 months, and analyzed using the described HPLC method.

b. Results—HPLC Purity

Samples of 1α-hydroxyvitamin D₂ analyzed to have initial (t=0) HPLC a/a purity of >99.0% a/a were shown to have no reduction in purity below 99.0% under the conditions of long term storage, at any of the time-points of 1, 3, 6 and 9 months.

c. Results—HPLC Assay

Samples of 1α-hydroxyvitamin D₂ analyzed to have initial (t=0) HPLC w/w assay of >99.0% w/w were shown to have no reduction in HPLC assay below 99.0% w/w, under the conditions of long term storage, at any of the time-points of 1, 3, 6 and 9 months.

What is claimed is:

1. Stabilized 1α-hydroxyvitamin D₂ which is characterized by a purity of at least 99%, and by a degree of stability such that it exhibits no reduction in purity after storage for one month at 25±2° C. and 60±2% relative humidity under argon head space.

2. Stabilized 1α-hydroxyvitamin D₂ according to claim 1 further characterized by a degree of stability such that it exhibits no reduction in purity after storage for six months at 25±2° C. and 60±2% relative humidity under argon head space.

3. Stabilized 1α-hydroxyvitamin D₂ according to claim 1 further characterized by a degree of stability such that it exhibits no reduction in purity after storage for nine months at −20±5° C. in ICH stability studies, under argon head space.

4. Stabilized 1α-hydroxyvitamin D₂ according to claim 1 further characterized by a degree of stability such that it exhibits no reduction in purity after storage for nine months at 5±3° C. in ICH stability studies, under argon head space.

5. A process of preparing stabilized 1α-hydroxyvitamin D₂ of at least 99% purity, which comprises:
chromatographically purifying 1α-hydroxyvitamin D₂ monoacetate,
chemically removing the acetate protectant group from the purified product to form 1αa-hydroxyvitamin D₂,
and precipitating the 1α-hydroxyvitamin D₂ so formed from a mixed organic solvent consisting essentially of at least one C1-C6 dialkyl ether or C1-C6 alkyl ester, and at least one C5-C12 hydrocarbon.

6. A process according to claim 5 wherein the mixed organic solvent is tert.butyl methyl ether and heptane.

7. A process according to claim 6 wherein the mixed organic solvent comprises an excess v/v of heptane.

8. A process according to claim 7 wherein the mixed solvent comprises about 3:1 v/v of heptane to MTBE

9. A process according to claim 5 wherein the 1α-hydroxyvitamin D₂ monoacetate is prepared by treating 1-OH-cyclovitamin D2 with acetic acid at elevated temperature.

10. A process according to claim 5 wherein the chemical removal of the acetate protectant group is conducted at room temperature.
